(19) United States
(12) Patent Application Publication
(10) Pub. No.: US 2014/0364708 A1
(43) Pub. Date: Dec. 11, 2014

(54) APPARATUS FOR ENHANCING THE MOLD-IN ALGORITHM

(71) Applicant: Wei-Kung Wang, Taipei (TW)

(72) Inventor: Wei-Kung Wang, Taipei (TW)

(21) Appl. No.: 14/470,275

(22) Filed: Aug. 27, 2014

Related U.S. Application Data
(63) Continuation-in-part of application No. 12/199,769, filed on Aug. 27, 2008.
Application No. 12/199,769 is a continuation-in-part of application No. 10/123,124, filed on Apr. 16, 2002, now abandoned.
Application No. 10/123,124 is a continuation-in-part of application No. 09/766,237, filed on Jan. 19, 2001, now abandoned.

(30) Foreign Application Priority Data
Mar. 17, 2000 (TW) 89104938

(51) Int. Cl. A61B 5/00; A61B 5/145; A61B 5/1455

(52) U.S. Cl. CPC A61B 5/7203; A61B 5/1455; A61B 5/14532; A61B 5/702
USPC 600/316; 600/322

(57) ABSTRACT

Disclosed is an apparatus for measuring the indicators of a specific ingredient in a solution. According to one embodiment of the present invention, said apparatus comprises: a signal collector for collecting a plurality of signals from a target in a selected volume of the solution. Beam splitters for splitting said signals and several designs for generating and transmitting the signals to the detectors. After the signal's detection, a mold-in algorithm is used to remove the noise. The filtered signals are used to get several indicators, which are correlated to the concentration of ingredients in the solution. This apparatus is designed to enhance the power of the mold-in algorithm. The present invention provides an apparatus for effectively measuring in-situ without the need of extracting the solution out of its original container.

### CROSS-REFERENCE TO RELATED APPLICATIONS

The present application is a continuation-in-part application of a pending U.S. patent application Ser. No. 12/199,769, entitled “APPARATUS FOR MEASURING CONCENTRATION OF A SPECIFIC INGREDIENT IN-SITU” and filed on Aug. 27, 2008, which is a continuation-in-part of a U.S. patent application Ser. No. 10/123,124, entitled “APPARATUS FOR MEASURING CONCENTRATION OF A SPECIFIC INGREDIENT IN-SITU” and filed on Apr. 16, 2002 (now abandoned), which is a continuation-in-part of a U.S. patent application Ser. No. 09/766,237, entitled “MOLD-IN METHOD AND APPARATUS” and filed on Jan. 19, 2001 (now abandoned) by the same inventor of the present application, and claims the benefit thereof and incorporates the same by reference.

### FIELD OF THE INVENTION

The present invention relates to an apparatus for measuring concentration of a specific ingredient in-situ through enhancing efficiency of a mold-in algorithm.

### BACKGROUND AND SUMMARY OF THE INVENTION

To measure concentration of an ingredient in a solution is usually to have the solution extracted from its container and put into a test tube or a cuvette, which is another container with a known volume (or more precisely, a known signal path). After a specific signal generated from the specific ingredient is measured, together with the known volume, the concentration can be determined by the ratio of the amount of the ingredient to the volume.

However, if such measurement is to be taken an in-situ (i.e., the solution had better not be extracted from the container such as cases of extracting blood from a blood vessel or moving a sample out of a production line), other information is required to determine the concentration.

Therefore, for the case of getting correlation to the concentration of one ingredient, at least two signals are needed. We shall call a calculated result of the signals from a source that contains the same ingredient as an indicator of the ingredient.

### BRIEF DESCRIPTION OF THE DRAWING

The present invention can be better understood through the accompanying drawing in which:

FIG. 1 shows an apparatus for measuring the concentration of a specific ingredient in-situ, according to one embodiment of the present invention.

### DETAILED DESCRIPTION OF THE INVENTION

In the figured embodiment, an optical signal (emanation or induced signal) is used as an example.

To accurately measure at least two signals from the same ingredient, the signals are required to be obtained from the same tissue. Particularly, the signals are induced by an input signal from an input signal source, the input signal source should be incident on the same tissue, and then result data are collected from targets through a signal collector.

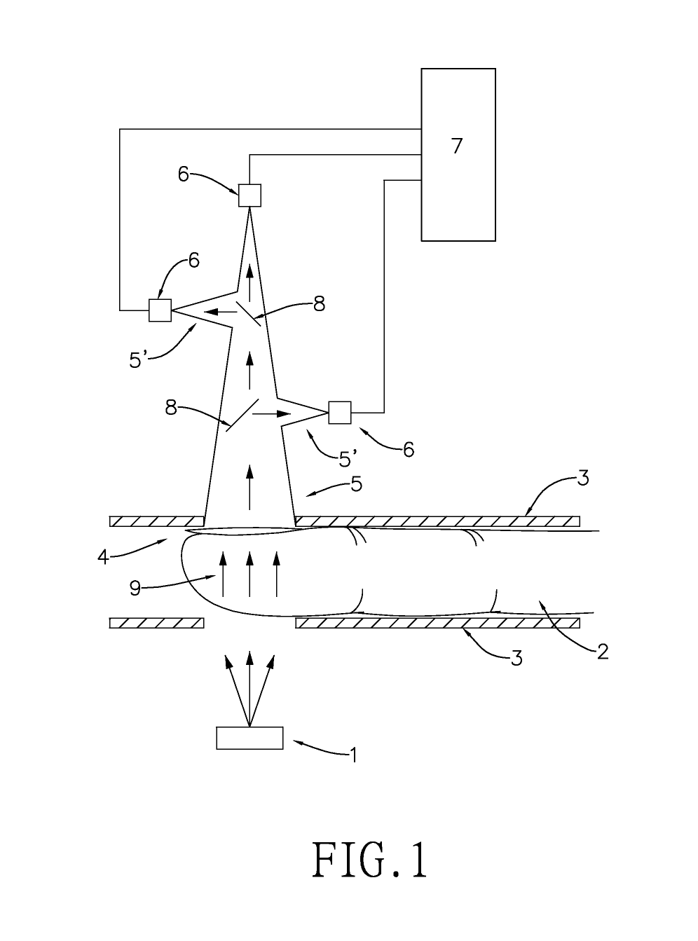

With reference to FIG. 1, to ensure a measured volume is the same during one measurement and the following measurement, a subject clamp, such as an envelope 3, is used to fix a position of a subject, such as a finger 2, and a position fixing tool, disclosed in U.S. patent application Ser. No. 14/327,485, can be introduced to ensure the same measured volume, for monitoring.

After the signals are collected, a spectroscopic method is needed to separate the at least two signals and collect the signals as much as possible. A conventional way is to use grating. According to the exemplary apparatus of the present invention shown in FIG. 1, two small cones 5′ and a large cone 5 housing two dichroic beam splitters 8 are used as the signal collector to ensure a better collection of the signals from the tissue.

As shown in FIG. 1, the signals are collected from the finger 2. Light from a homogenous light source 1 is incident into the inner side of the finger 2 through a signal guide (not shown in the figure) that has an adjustable aperture that can change position, shape and size as well as optical fiber or lenses. It will direct the signal from the light source to a selected volume. After being interactive with the finger 2, the light 9 comes out from a nail side 4 of the finger 2 and is collected by the large cone 5. The finger 2 is clamped by an engulfed structure such as the envelope 3 to fix the position of the finger 2 to be investigated. Both the signal guide and the signal collector are attached to the envelope 3. The signal collector has an adapter with an adjustable aperture that can change position, shape and size as well as some optical fiber or lenses. It will collect the signal from a selected volume.

In order to detect the concentrations of other ingredients in the blood, other specific signals, for example, signals of uric acid, cholesterol, triglycerol, hemoglobin or any drugs or ingredients are detected. Such signals can be detected one at a time by using the measurement apparatus shown in FIG. 1. Or, several ingredients can be detected at the same time. In the latter case, the dichroic beam splitters 8 are needed to separate the signals, and each of the cones 5, 5′ (including one large cone 5 and several small cones 5′) has lens to collect and focus each of the signals into corresponding designated detectors 6. The detectors 6, which are connected to a processing circuit 7, are set at tips of the cones 5, 5′ so as to collect the signals. A monochrometer that includes a band pass filter can be used to further refine a spectrum in each cone 5, 5′. Inner surfaces of the cones 5, 5′ are made highly reflective with reflection rate of 80% or above, and the higher the better, to increase their ability to collect the signals.

The processing circuit 7 uses a mold-in algorithm to remove noise, so that the filtered signals can be used to generate accurate indicators for the ingredients. The signal source 1 is a homogenous light source, and gives the same spectrum in every direction. This will further enhance the power of the mold-in algorithm.

A pulsatile oxygenometer can be one of the examples for such use, the signals of hemoglobin and oxyhemoglobin are used to get an indicator for the blood oxygen content.

The other example is blood glucose. As the signals of glucose-hemoglobin aggregates, the signal with oxygen and the signal without oxygen are filtered, so as to get an indicator for the blood glucose. The rule can be generalized as all the signals from signal sources that contain the specific ingredient are collected and analyzed to construct an algorithm to find an indicator for this specific ingredient and a correlation bet, the indicator and the concentration of a specific ingredient.

To make sure the indicator is accurate, we need to remove the noise as much as possible, and the best way to filter out the noise in a pulsatile container is the mold-in algorithm we disclosed in U.S. Pat. No. 7,389,132 B2. This invention can be viewed as the hardware designed especially for the mold-in algorithm. To improve the power of the mold-in algorithm, the signal source 1 to put into the subject, i.e. the finger 2, is one source. If multiple sources such as several LEDs (light emitting diodes) are used, they need to be well mixed before they are led to the target as one homogenous light to improve the usefulness of the mold-in algorithm.

The induced signal can be change of intensity such as absorption or scattering, or the change of signal property such as fluorescent.

The signals could be emanations such as α, β or γ particles emitted from isotopes decay, or chemi-luminance-light emitted by chemical energy. The signals could also be secondary or induced signals such as transmittance, scattering, fluorescence, Raman, etc., induced by another electromagnetic (EM) wave such as X-ray, visible, ultra-violet, infrared or microwave. To generate EM wave, all kinds of laser, diode laser, light emitted diode, lamps or EM sources can be used. They must be homogenized to be more useful.

For any induced signals, there is always a time delay from excitation to emission of the induced signal. The incident signal could be guided at an earlier time to excite the target in a selected volume to be measured. and after time At, the induced signal is collected. This method is referred to as “time resolved technique.” The technique can be used in the exemplary apparatus for reducing noise. The technique will be more useful when the excited target is moving. Assume the target is at position x with a velocity V*. After Δt, the excited target will move to x+V*Δt and emits the induced signal at this position. The target can be excited in a volume at position x, as time t, then the induced signal from the target in the specific volume is measured at x+Ax=x+V*Δt, at the time t+Δt. Thus, the noise resulted from the stationary (not moving) parts can be cut.

Signal-noise ratio can be improved by further using switches. When the switch of the guide for the input signal is on, the switch for the signal collector is off; when the switch of the guide for the input signal is off, the switch for the signal collector is on. Such on-off circle can be repeated for a lot of times to improve the signal-noise ratio. The above-mentioned arrangement is very useful as the targets are moving in a conduit such as an artery or production line.

As the invention thus described, it will be obvious that the embodiments and description are not intended to limit the invention. The invention may vary in many ways. Such variations are not to be regarded as a departure from the spirit and scope of the invention, and all such modifications, as would be obvious to one skilled in the art, are intended for inclusion within the scope of the following claims.

What is claimed is:

1. An apparatus for non-invasively measuring indicators of ingredients in a solution confined in an in-situ compartment comprising:
a first means for selecting a volume;
a signal collector for collecting signals from a target ingredient in a selected volume of the solution;
a means for detecting said signals from the target ingredient;
a means for separating said signals and transmitting said separated signals to said detecting means; and
a means for using a mold-in algorithm to remove noise out of the signals, and calculating the indicators of the ingredients.

2. The apparatus according to claim 1, wherein
said apparatus further comprises an input signal source; and
said signals collected comprise induced signals from an input signal from the input signal source, and a second means for selecting the volume by directing the input signal to the volume.

3. The apparatus according to claim 1, wherein said detecting means comprises a plurality of detectors respectively located at tips of a plurality of cones.

4. The apparatus according to claim 1, wherein said separating means comprises a dichroic beam splitter.

5. The apparatus according to claim 3, wherein each said plurality of cones comprises a lens for focusing the collected signal toward a corresponding detector.

6. The apparatus according to claim 3, wherein said plurality of cones comprises a highly reflective surface with reflection rate of 80% or above.

7. The apparatus according to claim 2, wherein said signal collector comprises an adapter for collecting said signals from said selected volume.

8. The apparatus according to claim 7, wherein said first means for selecting the volume comprises a signal guide for directing said input signal into a selected volume of the target.

9. The apparatus according to claim 8, wherein said signal guide comprised in said first means for selecting the volume directs said input signal into said target in said selected volume V at time t, and said signal collector collects said signals from another selected volume V′;
wherein V′ is a distribution of said target at time t=t+Δt.

10. The apparatus according to claim 9, wherein said target moves with a velocity V*, and said V′ is a linear transition from V to V+V*t.

11. The apparatus according to claim 10, wherein each of said signal guide and said signal collector comprises a switch.

12. The apparatus according to claim 11, wherein the switch of said signal collector is open after a predetermined period of time when the switch of said signal guide is closed.

13. The apparatus according to claim 12, wherein said switches are changed between open and closed for a plurality of times.

14. The apparatus according to claim 1, wherein said first means for selecting the volume further comprises an envelope and a position fixing device for monitoring the indicators and for repeated measurement.

15. The apparatus according to claim 1, wherein said signals after removing the noise are arranged and analyzed according to the ingredient that generates said signals to construct the indicator for said ingredient.

16. The apparatus according to claim 2, wherein said signals after removing the noise are arranged and analyzed according to the ingredient that generates said signals to construct the indicator for said ingredient.

17. The apparatus according to claim 2, wherein said indicator is for blood glucose.

18. The apparatus according to claim 2, wherein said input signal is homogeneous light source.
